(19) United States
(12) Patent Application Publication
(10) Pub. No.: US 2021/0315733 A1
(43) Pub. Date: Oct. 14, 2021

(54) Cooling Sheet

(71) Applicant: Sung Ah Cho, Seongbuk-gu Seoul (KR)

(72) Inventor: Sung Ah Cho, Seongbuk-gu Seoul (KR)

(21) Appl. No.: 16/490,542

(22) PCT Filed: Apr. 25, 2019
(86) PCT No.: PCT/KR2019/005032

§ 371 Date: Sep. 1, 2019

(30) Foreign Application Priority Data
May 9, 2018 (KR) 10-2018-0053265

(51) Int. Cl. A61F 7/02

(52) U.S. Cl. CPC A61F 7/02; A61F 2007/0011; A61F 2007/0292; A61F 2007/026

(57) ABSTRACT

A cooling sheet is provided. The cooling sheet according to one embodiment of the present invention includes a moisture storage layer which stores therein moisture, an absorbing layer which is fused to one surface of the moisture storage layer to absorb the moisture and is applied with microcapsules containing a functional material, and a water repellent layer which is fused to the other surface of the moisture storage layer to be in contact with skin, prevents the moisture from escaping and absorbs air from outside. Accordingly, it is possible to provide a cooling sensation using heat of evaporation when in contact with skin while preventing moisture from escaping to the outside along seam lines.

### BACKGROUND

### 1. Field

The following description relates to a cooling sheet, and more specifically, to a technology for providing a cooling sensation when worn on the skin of a person or pet.

### 2. Description of Related Art

Work or various outdoor activities on a hot summer day cause sweating. Such sweat flows especially on a neck and back, and a large amount of sweat makes clothes stick to the neck and back, which causes discomfort. When the clothes stick to the neck or back due to the sweat, air permeability is degraded and thus the body temperature rises, leading to more sweating.

As one of prior arts, Japanese Patent Publication No. 9-253119 (published on Sep. 30, 1997) discloses a cooling product that can be used in a neck band, a wrist band, or the like, by injecting super absorbent resin into a tube-like structure formed of a fabric. This prior art discloses a technique of sewing a number of small tubes to be connected to each other in order to overcome a drawback in that super absorbent resin inside a sewn fabric product in the form of a single tube swells when absorbing water and a cross-section becomes almost circular, which causes a reduced area in contact with skin.

As the super absorbent resin swells, the tubular shape becomes circular, and hence increase of skin contact area is insufficient as compared to a plane, and there is a limitation in that water escapes through a sewn surface, causing discomfort.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

The present invention is intended to provide a cooling sheet capable of providing a cooling sensation using heat of evaporation when in contact with the skin while preventing moisture from escaping to the outside along seam lines.

In addition, the present invention is intended to provide a cooling sheet capable of increasing a cooling sensation on the skin by promoting thermal circulating using a fiber block with a double structure.

Further, the present invention is intended to provide a cooling sheet capable of improving a cooling sensation effect by providing a volatile liquid coolant using a cooling capsule and thereby increasing the evaporation rate.

In one general aspect, there is provided a cooling sheet including a moisture storage layer which stores therein moisture, an absorbing layer which is fused to one surface of the moisture storage layer to absorb the moisture and is applied with microcapsules containing a functional material, and a water repellent layer which is fused to the other surface of the moisture storage layer to be in contact with skin, prevents the moisture from escaping and absorbs air from outside.

The moisture storage layer may be formed by vertically stacking a plurality of fiber blocks spaced apart from each other wherein the fiber blocks are formed by polymer-bonding super absorbent fiber powders.

The cooling sheet may further include a first hot-melt layer interposed and thermal fused between the moisture storage layer and the absorbing layer and a second hot-melt layer interposed and thermal fused between the moisture storage layer and the water repellent layer.

The water repellent layer may allow heat of the skin to be exhausted through heat radiating grooves on one surface thereof which are not in contact with the skin.

The moisture storage layer may include a first moisture storage layer formed by a plurality of first fiber blocks having a first absorption rate and spaced apart from each other and a second moisture layer formed by a plurality of second fiber blocks which have a second absorption rate and are spaced apart from each other and stacked at positions corresponding to spaces between the first fiber blocks.

Other features and aspects will be apparent from the following detailed description, the drawings, and the claims.

### BRIEF DESCRIPTION OF THE DRAWINGS

FIG. 1 is a diagram illustrating a usage state of cooling sheet according to one embodiment of the present invention.

FIG. 2 is a diagram illustrating a configuration of cooling sheet according to one embodiment of the present invention.

FIG. 3 is a diagram illustrating a detailed configuration of the cooling sheet shown in FIG. 2.

FIG. 4 is a diagram for describing heat exchange occurring in the cooling sheet of FIG. 2.

FIG. 5 is a diagram for describing forming of heat radiating grooves on the cooling sheet of FIG. 2.

FIG. 6 is a diagram for describing an electronic tag further included in the cooling sheet of FIG. 2.

FIG. 7 is a diagram for describing a cooling capsule further included in the cooling sheet of FIG. 2.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

Hereinafter, in order to facilitate understanding and reproduce by those skilled in the art, the present invention will be described in detail by explaining exemplary embodiments with reference to the accompanying drawings. Terms described in below are selected by considering functions in the embodiment and meanings may vary depending on, for example, a user or operator's intentions or customs. Therefore, in the following embodiments, when terms are specifically defined, the meanings of terms should be interpreted based on definitions, and otherwise, should be interpreted based on general meanings recognized by those skilled in the art.

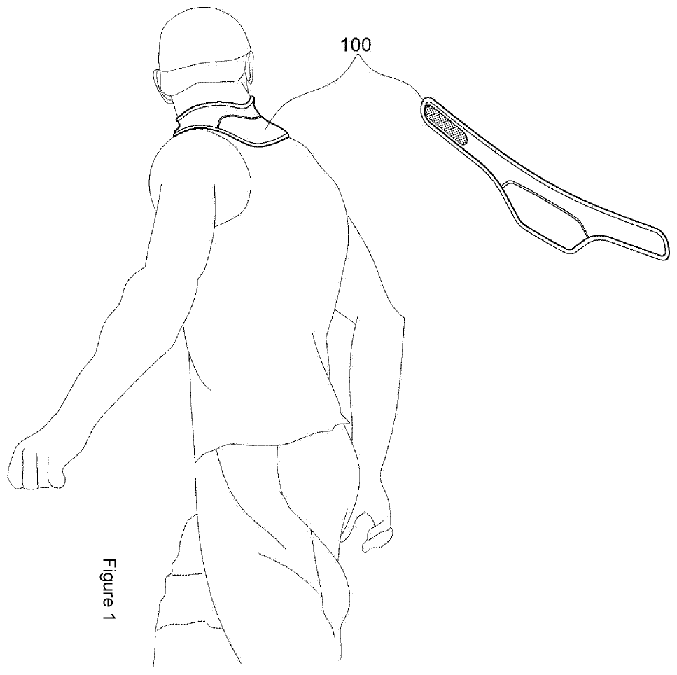

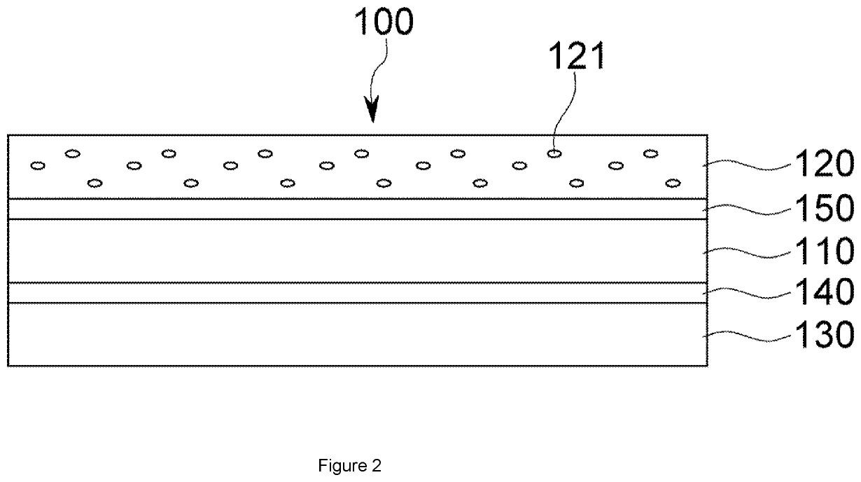

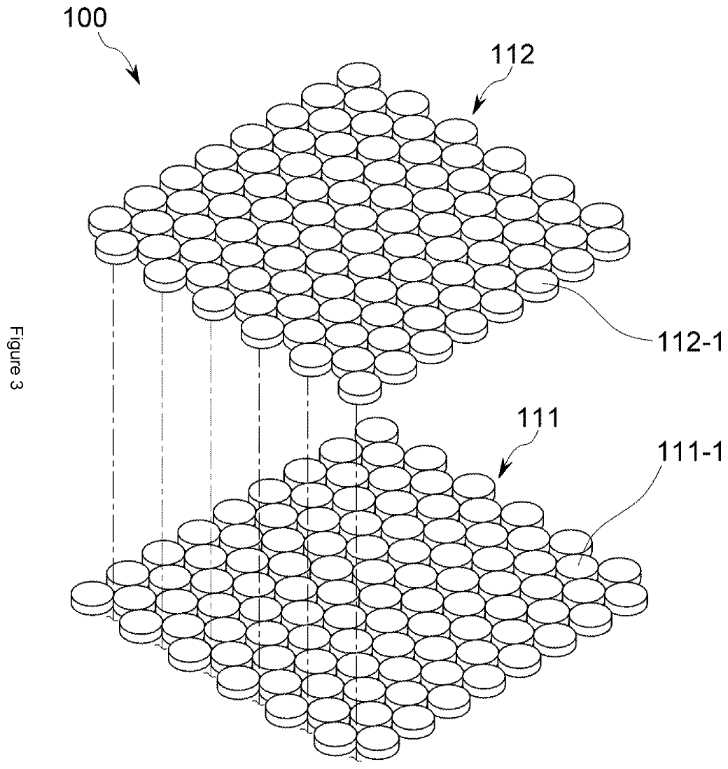

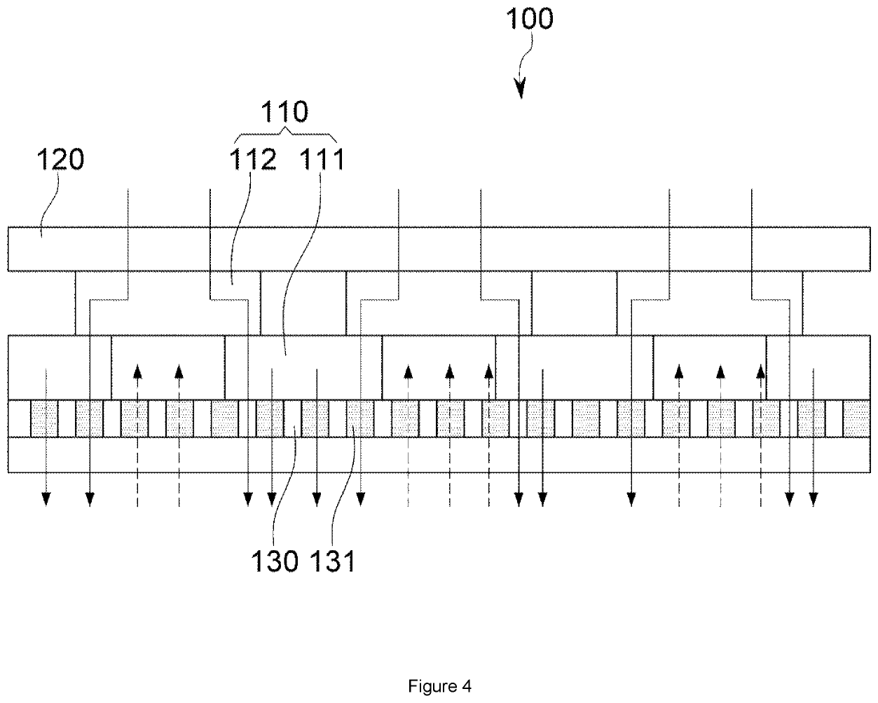

FIG. 1 is a diagram illustrating a usage state of cooling sheet according to one embodiment of the present invention, FIG. 2 is a diagram illustrating a configuration of cooling sheet according to one embodiment of the present invention, FIG. 3 is a diagram illustrating a detailed configuration of the cooling sheet shown in FIG. 2, and FIG. 4 is a diagram for describing heat exchange occurring in the cooling sheet of FIG. 2.

Referring to FIGS. 1 to 4, the cooling sheet 100 according to one embodiment of the present invention may be in the form of a band that can be wrapped around a neck or wrist of a person or may be used in a variety of goods, such as a vest, a cap, and the like. In addition, the cooling sheet may be used for pet clothing or the like. Specifically, the cooling sheet 100 includes a moisture storage layer 110, an absorbing layer 120, and a water repellent layer 130.

The moisture storage layer 110 is made of a fiber material and stores therein moisture. The moisture storage layer 110 includes a high-absorbent fiber material to maintain a large amount of moisture for a long time. The moisture storage layer 110 may use natural fibers or chemical fibers, and may also use non-woven fabrics, but is not necessarily limited thereto. The absorbing layer 120 is formed on one surface of the moisture storage layer 110 and the water repellent layer 130 is formed on the other surface. The moisture storage layer 110 stores therein moisture entering through the absorbing layer 120. The moisture storage layer 110 receives heat to generate evaporation heat when the water repellent layer 130 is in contact with the skin of a person. In addition, the moisture of the moisture storage layer 110 is evaporated as the moisture of the absorbing layer 120 is evaporated by sunlight or wind from the outside. Accordingly, the moisture stored in the moisture storage layer 110 lowers skin temperature as it is evaporated, thereby providing a cooling sensation.

In addition, the moisture storage layer 110 may be formed by vertically stacking a plurality of fiber blocks 111-1 and 112-1 spaced from each other, wherein the fiber blocks 111-1 and 112-1 are formed by polymer-bonding powders or fibers of a super absorbent fiber material. The plurality of fiber blocks 111-1 and 112-1 may be formed in a cylindrical shape by pressing super absorbent fiber powders, but is not necessarily limited thereto. More specifically, the moisture storage layer 110 may include a first moisture storage layer 111 and a second moisture storage layer 112. The first moisture storage layer 111 is formed by a plurality of first fiber blocks 111-1 having a first absorption rate and spaced apart from each other. In this case, the first absorption rate of the first fiber blocks 111-1 may be altered according to compressed density of material.

The second moisture storage layer 112 is formed by a plurality of second fiber blocks 112-1 having a second absorption rate and spaced apart from each other. In this case, the second absorption rate of the second fiber blocks 112-1 may be altered according to a compressed density or material and may be different from the absorption rate of the first fiber blocks 111-1. The second moisture storage layer 112 is formed by vertically stacking the plurality of second fiber blocks 112-1 spaced apart from each other, wherein the plurality of second fiber blocks 112-1 are stacked at positions corresponding to spaces between the plurality of first fiber blocks 111-1. In this case, the first moisture storage layer 111 and the second moisture storage layer 112 are stacked over each other in such a manner that predetermined portions thereof are in contact with and connected to each other and thereby spaces are formed therebetween. The first moisture storage layer 111 absorbs heat of skin from the water repellent layer 130 and the absorbed heat is evaporated through the spaces between the plurality of spaced second fiber blocks 112-1 of the second moisture storage layer 112. Accordingly, the evaporation of the moisture absorbed into the moisture storage layer 110 is facilitated, so that the cooling sensation effect may be increased.

The absorbing layer 120 may be fused to one surface of the moisture storage layer 110 to absorb moisture. In this case, the absorbing layer 120 may be fused by at least one of thermal fusion, ultrasonic fusion, and high frequency fusion. The absorbing layer 120 may be partially fused to the moisture storage layer 110 to hermitically seal an outer portion of the moisture storage layer 110, rather than being fused over the entire surface the moisture storage layer 110. The absorbing layer 120 may be made of a material having sweat absorption and quick drying functions of absorbing moisture and rapidly drying. The absorbing layer 120 may be preferably formed by including a component capable of blocking ultraviolet rays. The absorbing layer 120 absorbs moisture and serves to evaporate the moisture. In this case, the moisture absorbs heat of the skin as it is vaporized. In this case, the absorbing layer 120 may be preferably formed as a microstructure that can quickly absorb moisture.

In addition, the absorbing layer 120 may be coated with a functional material with antimicrobial, aromatic components, such as phytoncide, silver nanoparticles, charcoal, menthol, mint, and xylitol, in the form of microcapsules. In addition, an herbal material with pest-repellent components may be coated in the form of microcapsules. The type of material to be coated may be varied according to user's settings. A reflective laminate sheet that reflects light is formed on one side of the surface of the absorbing layer 120. This may be formed in a shape, such as a symbol, a character, or a figure, to reflect light at night. Velcro may be provided on the other side of the surface of the absorbing layer 120, but is not necessarily limited thereto such that a coupling means in various forms may be applied. Thermochromic ink may be applied to the surface of the absorbing layer 120 so that the color of the ink may vary or letters may appear according to a temperature of moisture. Accordingly, the user is allowed to visually confirm the temperature of the cooling sheet 100 itself and the time for recharging with moisture.

The water repellent layer 130 is fused to the other surface of the moisture storage layer 110 and is waterproof. In this case, the water repellent layer 130 may be fused by at least one of thermal fusion, ultrasonic fusion, and high frequency fusion. The water repellent layer 130 may be partially fused to hermitically seal the outer portion of the moisture storage layer 110, rather than being fused over the entire surface of the moisture storage layer 110. The water repellent layer 130 prevents the moisture absorbed into the moisture storage layer 110 from being in direct contact with the skin but enables heat exchange by passing air therethrough. The water repellent layer 130 may be preferably formed in a predetermined thickness. One surface of the water repellent layer 130 is bonded to the moisture storage layer 110 and the other surface is brought into contact with the skin of a person. Velcro may be provided on one side of the surface of the water repellent layer 130 and the water repellent layer 130 may be fixed via the Velcro thereof which is coupled to the Velcro provided on the absorbing layer 120.

Referring to FIG. 4, the cooling sheet 100 according to one embodiment of the present invention is provided with a plurality of vent holes 131 formed on the water repellent layer 130 to enable heat exchange. The vent hole 131 has a micropore structure and is preferably formed in a size that allows gas but not liquid to pass. The heat of the skin transferred from the water repellent layer 130 is exhausted to the outside through the moisture storage layer 110 and the absorbing layer 120. In this case, the water repellent layer 130 may be formed of an elastic member. This is to enable the cooling sheet 100 to be wrapped around a wrist or neck without a separate coupling means. The water repellent layer 130 is preferably formed in a predetermined thickness.

In addition, when the moisture storage layer 110 is formed in a double structure of the first moisture storage layer 111 and the second moisture storage layer 112, heat moves through the space between the layers and thereby heat exchange may be performed quickly. Air of a relatively low temperature moves from the absorbing layer 120 to the skin through the moisture storage layer 110 and the water repellent layer 140. Air of a relatively high temperature is exhausted from the water repellent layer 130 through the moisture storage layer 110 and the absorbing layer 120.

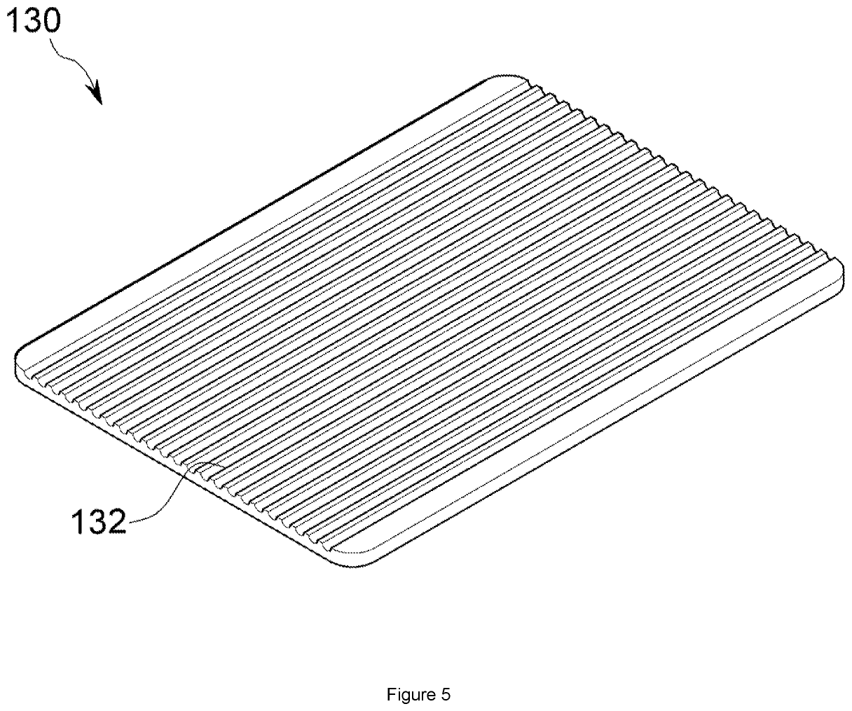

FIG. 5 is a diagram for describing forming of heat radiating grooves on the cooling sheet of FIG. 2.

Referring to FIG. 5, heat radiating grooves 132 may be formed on a surface of the water repellent layer 130 in contact with the skin. The water repellent layer 130 is brought into contact with the skin and the heat radiating grooves 132 are formed concavely and thus are not in contact with the skin. Heat of the skin is transferred to the water repellent layer 130 in contact with the skin and is absorbed by the moisture storage layer 110. In addition, the heat of the skin is exhausted through the heat radiating grooves 132 that are not in contact with the skin.

Meanwhile, the cooling sheet 100 according to one embodiment of the present invention may further include a first hot-melt layer 140 and a second hot-melt layer 150.

The first hot-melt layer 140 is interposed and thermal fused between the moisture storage layer 110 and the absorbing layer 120. The first hot-melt layer 140 is for bonding between the moisture storage layer 110 and the absorbing layer 120 without sewing. The first hot-melt layer 140 is bonded by heat, ultrasound, high frequency, or the like. The first hot-melt layer 140 bonds outer surfaces of the moisture storage layer 110 and the absorbing layer 120. The first hot-melt layer 140 may be provided in the form of a film sheet, but is not necessarily limited thereto. The first hot-melt layer 140 maintains the bonding between the moisture storage layer 110 and the absorbing layer 120 and prevents escape of moisture through the outer surface of the moisture storage layer 110.

The second hot-melt layer 150 is interposed and thermal fused between the moisture storage layer 110 and the water repellent layer 130. The second hot-melt layer 150 is for bonding between the moisture storage layer and the water repellent layer 130 without sewing. The second hot-melt layer 150 is bonded by heat, ultrasound, high frequency, or the like. The second hot-melt layer 150 bonds outer surfaces of the moisture storage layer 110 and the water repellent layer 130. The second hot-melt layer 150 may be provided in the form of a film sheet, but is not necessarily limited thereto. The second hot-melt layer 150 maintains the bonding between the moisture storage layer 110 and the water repellent layer 130 and prevents escape of moisture through the outer surface of the moisture storage layer 110.

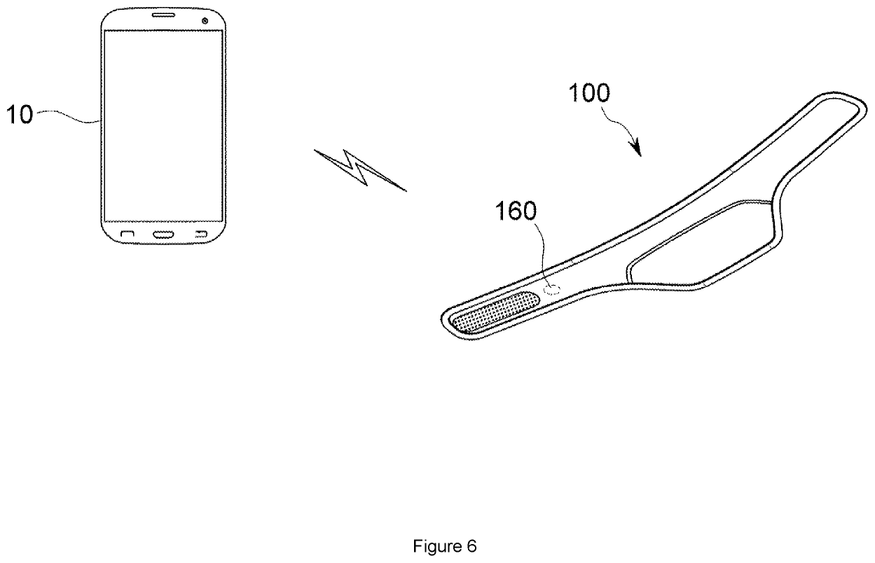

FIG. 6 is a diagram for describing an electronic tag further included in the cooling sheet of FIG. 2.

Referring to FIG. 6, the cooling sheet 100 according to one embodiment of the present invention may further include an electronic tag 160. The electronic tag 160 may be formed of a waterproof material and interposed between the absorbing layer 120 and the water repellent layer 130. The electronic tag 160 may be provided in the form of a radio frequency (RF) tag, a near field communication (NFC) tag, or a beacon, but is not necessarily limited thereto. The electronic tag 160 may communicate with a user terminal 10. When equipped with a temperature sensor or a moisture sensor, the electronic tag 160 may transmit temperature information or moisture content information of the moisture storage layer 110 in the cooling sheet 100 to the user terminal 10. This is to inform the user so that the user can replenish moisture.

In addition, the electronic tag 160 may be provided in the form of a Peltier element. In this case, the electronic tag 160 may be supplied with power using a rechargeable built-in battery (not shown) or using a solar cell (not shown) formed on the surface of the absorbing layer 120. When the electronic gat 160 is formed as a Peltier element, cooling or heating may be performed according to current control. In this case, temperature may be adjusted through the electronic tag 160 by use of the user terminal 10. When the cooling sheet 100 is used for pet clothing or baby clothing, a guardian may be allowed to remotely adjust temperature through the user terminal 10 or to be notified of whether moisture replenishment is required.

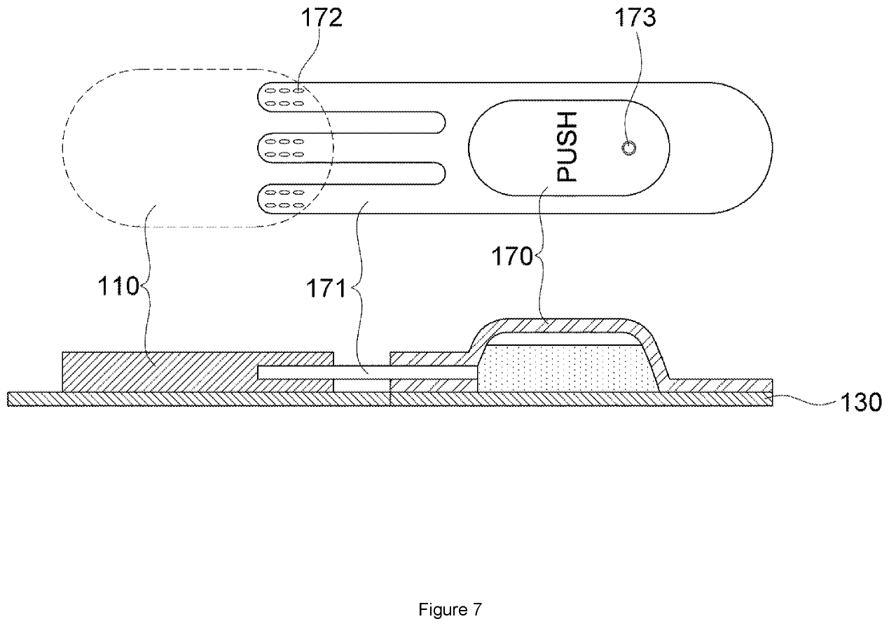

FIG. 7 is a diagram for describing a cooling capsule further included in the cooling sheet of FIG. 2.

Referring to FIG. 7, the cooling sheet 100 according to one embodiment of the present invention may further include a cooling capsule 170. The cooling capsule 170 has one side protruding toward a perforated region of the absorbing layer 120 and the other side thereof is connected to the moisture storage layer 110. A volatile liquid coolant, such as alcohol, may be filled inside the cooling capsule 170. A plurality of capillary tubes 171 are connected to the moisture storage layer 110 and the cooling capsule 170 transfers the liquid coolant to the moisture storage layer 110 through discharge holes 172. When the cooling capsule 170 is pressurized, the liquid coolant moves to the moisture storage layer 110 through the tubes 171. Before an external force is applied to the cooling capsule 170, the liquid coolant does not move to the tube 171 due to a pressure difference. When the liquid coolant is absorbed into the moisture storage layer 110, heat exchanged is promoted by faster volatility than that of water, thereby maximizing a cooling effect. In this case, one side of the cooling capsule 170 is provided with an inlet 173 through which the liquid coolant is filled for use.

As described above, it is possible to provide a cooling sensation using heat of evaporation when in contact with the skin while preventing moisture from escaping to the outside along seam lines.

In addition, heat exchange is promoted by using fiber blocks of a double structure so that a cooling sensation on the skin can be increased.

Further, a volatile liquid coolant is provided using the cooling capsule to increase the evaporation rate, thereby improving a cooling sensation effect.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

### REFERENCE NUMERALS

100: COOLING SHEET
110: MOISTURE STORAGE LAYER
111: FIRST MOISTURE STORAGE LAYER
111-1: FIRST FIBER BLOCK
112: SECOND MOISTURE STORAGE LAYER
112-1: SECOND FIBER BLOCK
120: ABSORBING LAYER
121: MICROCAPSULE
130: WATER REPELLENT LAYER
131: VENT HOLE
132: HEAT RADIATING GROOVE
140: FIRST HOT-MELT LAYER
150: SECOND HOT-MELT LAYER
160: ELECTRONIC TAG
170: COOLING CAPSULE
171: TUBE
172: DISCHARGE HOLE
173: INLET

What is claimed is:

1. A cooling sheet comprising:
a moisture storage layer which stores therein moisture;
an absorbing layer which is fused to one surface of the moisture storage layer to absorb the moisture and is applied with microcapsules containing a functional material; and
a water repellent layer which is fused to the other surface of the moisture storage layer to be in contact with skin, prevents the moisture from escaping and absorbs air from outside.

2. The cooling sheet of claim 1, wherein the moisture storage layer is formed by vertically stacking a plurality of fiber blocks spaced apart from each other wherein the fiber blocks are formed by polymer-bonding super absorbent fiber powders.

3. The cooling sheet of claim 1, further comprising:
a first hot-melt layer interposed and thermal fused between the moisture storage layer and the absorbing layer; and
a second hot-melt layer interposed and thermal fused between the moisture storage layer and the water repellent layer.

4. The cooling sheet of claim 1, wherein the water repellent layer allows heat of the skin to be exhausted through heat radiating grooves on one surface thereof which are not in contact with the skin.

5. The cooling sheet of claim 2, wherein the moisture storage layer includes
a first moisture storage layer formed by a plurality of first fiber blocks having a first absorption rate and spaced apart from each other and
a second moisture layer formed by a plurality of second fiber blocks which have a second absorption rate and are spaced apart from each other and stacked at positions corresponding to spaces between the first fiber blocks.
